(19) United States
(12) Patent Application Publication
(10) Pub. No.: US 2006/0235500 A1
(43) Pub. Date: Oct. 19, 2006

(54) Optic fibre device

(76) Inventors: Peter Gibson, Randwick (AU); Miroslaw Mackiewicz, Ryde (AU); Bas Van Dijk, Mechelen (BE)

Correspondence Address: JAGTIANI + GUTTAG, 10363-A DEMOCRACY LANE, FAIRFAX, VA, 22030, US

(21) Appl. No.: 10/518,810

(22) PCT Filed: Jun. 30, 2003
(86) PCT No.: PCT/AU03/00839

§ 371 Date: Apr. 20, 2006

(30) Foreign Application Priority Data
Jun. 28, 2002 (AU) PS3225
Feb. 21, 2003 (AU) 2003900775

(51) Int. Cl. A61N 1/05

(52) U.S. Cl. 607/137; 607/55

(57) ABSTRACT

An electrode array or assembly that enables a surgeon to visualise a region of the body and particularly the cochlea during or prior to implantation of the array or assembly into the body so that, where necessary, appropriate action can be taken by the surgeon in accordance with any complications to the surgery that may arise. Also described is a probe that provides a surgeon with a means to visualise the cochlea prior to or during implantation of an electrode array assembly.

### FIELD OF THE INVENTION

The present invention relates to an implantable device and, in particular, to a device for use in visually inspecting a cochlea prior to and during its implantation with an implantable cochlear electrode assembly.

### BACKGROUND OF THE INVENTION

Hearing loss, which may be due to many different causes, is generally of two types, conductive and sensorineural. Of these types, conductive hearing loss occurs where the normal mechanical pathways for sound to reach the hair cells in the cochlea are impeded, for example, by damage to the ossicles. Conductive hearing loss may often be helped by use of conventional hearing aid systems, which amplify sound so that acoustic information does reach the cochlea and the hair cells.

In many people who are profoundly deaf, however, the reason for deafness is sensorineural hearing loss. This type of hearing loss is due to the absence of, or destruction of, the hair cells in the cochlea which transduce acoustic signals into nerve impulses. These people are thus unable to derive suitable benefit from conventional hearing aid systems, because there is damage to or absence of the mechanism for nerve impulses to be generated from sound in the normal manner.

It is for this purpose that cochlear implant systems have been developed. Such systems bypass the hair cells in the cochlea and directly deliver electrical stimulation to the auditory nerve fibres, thereby allowing the brain to perceive a hearing sensation resembling the natural hearing sensation normally delivered to the auditory nerve. U.S. Pat. No. 4,532,930, the contents of which are incorporated herein by reference, provides a description of one type of traditional cochlear implant system.

Cochlear implant systems have typically consisted of two key components, namely an external component commonly referred to as a processor unit, and an implanted internal component commonly referred to as a receiver/stimulator unit. Traditionally, both of these components have cooperated together to provide the sound sensation to an implantee.

The external component has traditionally consisted of a microphone for detecting sounds, such as speech and environmental sounds, a speech processor that converts the detected sounds and particularly speech into a coded signal, a power source such as a battery, and an external antenna transmitter coil.

The coded signal output by the speech processor is transmitted transcutaneously to the implanted receiver/stimulator unit situated within a recess of the temporal bone of the implantee. This transcutaneous transmission occurs through use of an inductive coupling provided between the external antenna transmitter coil which is positioned to communicate with an implanted antenna receiver coil provided with the receiver/stimulator unit. This communication serves two essential purposes, firstly to transcutaneously transmit the coded sound signal and secondly to provide power to the implanted receiver/stimulator unit. Conventionally, this link has been in the form of a radio frequency (RF) link, but other such links have been proposed and implemented with varying degrees of success.

The implanted receiver/stimulator unit typically included the antenna receiver coil that receives the coded signal and power from the external processor component, and a stimulator that processes the coded signal and outputs a stimulation signal to an intracochlea electrode assembly which applies the electrical stimulation directly to the auditory nerve producing a hearing sensation corresponding to the original detected sound.

Fundamental to the performance of any cochlear implant system has been the method in which the electrical stimulation is applied to the auditory nerve. It has been found that in order for these electrodes of the intracochlea electrode assembly to be effective, the magnitude of the currents flowing from these electrodes and the intensity of the corresponding electric fields, are a function of the distance between the electrodes and the modiolus. If this distance is relatively great, the threshold current magnitude must be larger than if the distance is relatively small. Moreover, the current from each electrode may flow in all directions, and the electrical fields corresponding to adjacent electrodes may overlap, thereby causing cross-electrode interference. In order to reduce the threshold stimulation amplitude and to eliminate cross-electrode interference, it is advisable to keep the distance between the electrode array and the modiolus as small as possible. This is most effectively executed by providing the electrode array in the shape which generally follows the shape of the modiolus to obtain maximum access to the desired auditory nerves. This also ensures that the delivery of the electrical stimulation to the auditory nerve is most effective as the electrode contacts are as close to the auditory nerves that are particularly responsive to selected pitches of sound waves.

In order to achieve this electrode array position close to the inside wall of the cochlea, the electrode needs to be designed in such a way that it assumes this position upon or immediately following insertion into the cochlea. This is a challenge as the array needs to be shaped such that it assumes a curved shape to conform with the shape of the modiolus and must also be shaped such that the insertion process causes minimal trauma to the sensitive structures of the cochlea. In this regard, it has been found desirable for the electrode array to be generally straight during the insertion procedure.

Several procedures have been adopted to provide an electrode assembly that is relatively straightforward to insert while adopting a curved configuration following insertion in the cochlea. In one case, a platinum wire stylet is used to hold a pre-curved electrode array in a generally straight configuration up until insertion. During insertion, the platinum stylet is withdrawn allowing the array to return to its pre-curved configuration.

In another development, a relatively straight electrode array is inserted into the cochlea in a conventional manner and a positioning element is used to fill the space behind the electrode array to force the electrode array into contact with the inner wall of the cochlea.

In a still further arrangement, a longitudinal element that is arranged on one side of the array and constructed to change its dimension on insertion can be utilised. For example, the longitudinal element could include a hydrogel such as polyacrylic acid (PAA) which expands after insertion by absorbing water from the cochlear fluid.

In developing such electrode array designs, it is of great importance that the design be constructed to minimise potential damage to sensitive structures in the cochlear upon insertion and placement.

Unfortunately, for individuals suffering from profound hearing loss, there may be some deformation or ossification of a portion or the entire length of the cochlea restricting access to some of the nerves. In such instances, it is not possible to insert the electrode array through the entire length of the cochlea and as such the technique of inserting the electrode array may need to be altered or another type of electrode array employed.

One difficulty that can be faced by surgeons attempting to implant an electrode array into a cochlea having a scala tympani duct that is at least partially blocked or ossified is that the surgeon is unaware of this problem until after an insertion attempt has been made. This may result in additional trauma and damage to the cochlea by attempting the initial insertion and the unnecessary use of a number of devices prior to diagnosing the actual problem.

The present invention is directed to a means to allow a surgeon to visualise the cochlea prior to or, preferably, during implantation of an electrode assembly so that, where necessary, appropriate action can be taken by the surgeon in accordance with any complications to the surgery that may arise. Visual inspection in this manner enables a surgeon to avoid damage of the delicate structure of the cochlea by manoeuvring the electrode assembly in a desired direction.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect, the present invention is an implantable tissue stimulating device comprising an elongate carrier member having a proximal end, a distal end, and a plurality of electrodes mounted thereon between said proximal and distal ends, the elongate carrier member having at least a first lumen extending at least partially therethrough, the elongate carrier member further including one or more optic fibres positioned along a length of said at least a first lumen.

The implantable tissue stimulating device is preferably a cochlear electrode array.

The one or more optic fibres are preferably removably positioned within the lumen such that they may be used when required.

The one or more optic fibres allow a user to illuminate and/or visualise an area of the cochlea during or prior to surgery. In this embodiment, the elongate carrier member preferably includes a plurality of optic fibres. At least one of these optic fibres may act to illuminate a surgical site whereas at least a further optic fibre may allow a surgeon to visualise a surgical site. Typically, the plurality of optic fibres extend along the entire length of the lumen from a first end to a second end wherein said second end is positioned adjacent the distal end of the elongate carrier member.

The elongate carrier member preferably has a first configuration selected to allow said elongate carrier member to be inserted into a cochlea, and at least a second configuration wherein said elongate carrier member is adapted to apply a preselected tissue stimulation with the electrodes, said elongate carrier member being made of a resiliently flexible first material.

The one or more optic fibres in addition to acting as means to illuminate and/or visualise a surgical site may also act as a stiffening element that biases said elongate carrier member into said first configuration. Removal of the stiffening element causes the elongate carrier member to assume its second configuration.

In another embodiment, the elongate carrier member may have a second lumen formed therein. A stiffening member such as a stylet may extend through said second lumen to bias the elongate carrier member into its first configuration.

Because the one or more optic fibres are removable within the at least a first lumen said lumen may act as a drug delivery channel following removal of the one or more optic fibres.

According to a second aspect, the present invention is an implantable tissue-stimulating device comprising:

an elongate carrier member having a plurality of electrodes mounted thereon and having a first configuration selected to allow said elongate carrier member to be inserted into an implantee's body, and at least a second configuration wherein said elongate carrier member is adapted to apply a preselected tissue stimulation with the electrodes, said elongate carrier member being made of a resiliently flexible first material; and

a stiffening element removably positionable within said elongate carrier member that biases said elongate carrier member into said first configuration;

wherein said stiffening element comprises one or more optic fibres.

In a preferred embodiment, the second configuration of the elongate carrier member is curved. More preferably, the elongate carrier member adopts a spiral configuration when in the second configuration.

According to a third aspect, the present invention is a cochlear implant electrode assembly device comprising:

an elongate carrier member having a plurality of electrodes mounted thereon and having a first configuration selected to allow said elongate carrier member to be inserted into an implantee's cochlea, and at least a second configuration wherein said elongate carrier member is curved to match a surface of said cochlea, said elongate carrier member being made of a resiliently flexible first material; and

a stiffening element removably positionable within said elongate carrier member that biases said elongate carrier member into said first configuration;

wherein said stiffening element comprises one or more optic fibres.

The elongate carrier member of the second and third aspects preferably extends from a proximal end to a distal end.

In one embodiment of aspects one to three, the elongate carrier member can have a resiliently flexible tip member extending forwardly from the distal end of the elongate carrier member. The tip member is preferably light permeable, and more preferably at least substantially transparent. In one embodiment, the tip member can be hemispherical in form. The diameter preferably gradually decreases to form a rounded end. The tip member preferably has a diameter of about 140 μm.

The tip member preferably acts as a lens and allows illumination and/or visualisation, via the optic fibres, of a region at least adjacent the tip member of the elongate carrier member. In one embodiment, the tip member acts as a planar convex lens, however, other lens types can be formed.

In one embodiment, the tip member is formed from a transparent silicone compound. With reference to the second and third aspects, the tip member can have a stiffness that is relatively less stiff than said stiffening element.

The tip member can further be formed of a material that is substantially the same or the same stiffness as the elongate carrier member. In another embodiment, the tip member can be formed of a material that is relatively less stiff than at least a portion of the elongate carrier member. In a further embodiment, the tip member can be formed of a material that undergoes a change in stiffness, preferably a decrease in stiffness, on insertion into the body of a subject, such as the cochlea.

In a further embodiment, the stiffness of the tip member can vary along at least a portion of its length from a proximal end adjacent the first end of the elongate carrier member to a distal end. In one embodiment, the stiffness of the tip member can vary over the entire length of the tip member or only a portion thereof. The stiffness can increase from the distal end to the proximal end. In one embodiment, the stiffness of the tip member over said portion or its length can increase gradually from its distal end towards to the proximal end. The increase in stiffness can be substantially smooth.

In a further embodiment, the tip member can be formed of the same material as the body of the elongate carrier member. In another embodiment, the tip member can be formed of a different material to that of the body of the elongate member.

The tip member can be formed separately to the elongate carrier member and mounted thereto. For example, the tip member can be adhered to the distal end of the elongate carrier member with a clear adhesive. In another embodiment, the tip member can be integrally formed with the elongate carrier member. The tip member can be formed from a silicone material.

According to a fourth aspect, the present invention is a cochlear implant electrode assembly device comprising an elongate carrier member having a proximal end, a distal end, and a plurality of electrodes mounted thereon between said proximal and distal ends, the elongate carrier member having a first configuration selected to allow said member to be inserted into an implantee's cochlea, and at least a second configuration wherein said elongate carrier member is curved to match a surface of said cochlea, said elongate carrier member being made of a resiliently flexible first material and having a lumen formed therein extending from or adjacent the proximal end to or adjacent the distal end and adapted to receive a stiffening element removably positionable within said elongate carrier member that biases said elongate carrier member into said first configuration, wherein said distal end of said elongate carrier member comprises a transparent tip member.

In the fourth aspect, the assembly device is preferably pre-packaged with a stiffening element positioned within the lumen of the member. The stiffening element is preferably a stylet comprising one or more optic fibres.

The elongate carrier member of the above aspects is preferably preformed from a plastics material with memory and is preformed to the second configuration. The distal end of the elongate carrier member is preferably firstly inserted into an implantee. In a preferred embodiment, the first configuration is preferably substantially straight. More preferably, the first configuration is straight. In a preferred embodiment the elongate carrier member preferably adopts a spiral configuration when in the second configuration. The second configuration may be achieved by removal of the stiffening element.

In a preferred embodiment, the elongate carrier member is formed from a suitable biocompatible material. The material can be a silicone, such as a flexible silicone elastomer Silastic. Silastic MDX 4-4210 is an example of one suitable silicone for use in the formation of the elongate carrier member. The elongate carrier member can also be formed from a polyurethane or other similar materials.

In another embodiment, the stiffening element can comprise a monolithic optic fibre stylet. The stiffening element can also comprise a stylet formed of a plurality of optic fibres.

The stiffening element can extend through a single lumen in the elongate carrier member. In one embodiment, the lumen for the stiffening element can be cylindrical and also can have an opening formed therein. The stiffening element can extend out of the opening allowing the stiffening element to be manipulated and removed from the lumen during or following insertion of the elongate carrier member.

In a fifth aspect, the present invention is a stiffening element for an implantable tissue-stimulating device characterised in that the stiffening element comprises one or more optic fibres.

In this aspect, the tissue-stimulating device is preferably a cochlear implant electrode assembly device.

In the fifth aspect, the stiffening element can comprise a monolithic optic fibre stylet. In another embodiment of this aspect, the stiffening element can comprise a stylet formed of a plurality of optic fibres.

In one embodiment, a leading end of the stiffening element can be connected to an optical fibre termination means. In this regard, the termination means typically receives a first end of the stylet. The termination means preferably includes a light source, eyepiece, and/or a camera lens mounted thereto. The termination means typically receives light output by the light source and directs this light through the one or more optic fibres to their leading ends. This light is then preferably able to pass through the transparent tip member of the elongate carrier member and illuminate at least the region adjacent the tip member.

The eyepiece and/or the camera lens preferably receive light reflected through the one or more optic fibres from an object illuminated by the light emitting from the leading end of the optical fibres. A magnifying means and/or focussing means can be incorporated, if necessary, into the termination means.

The camera lens is preferably part of a video camera that allows recordal of the image detected by the camera lens. When used in respect of the cochlear implant electrode assembly devices of aspects four and five, the video footage is preferably used in real time during insertion of the elongate carrier member into a recipient's cochlea but can also be used as a means of reviewing the surgical procedure following completion of the surgery.

The present invention provides a surgeon with a means to illuminate and visualise the region of the cochlea at least adjacent the distal end or the tip member of the elongate carrier member as it is implanted in the cochlea. This provides the surgeon with a means to illuminate and visualise unexpected bone or tissue growth that might cause deflection of the member during its insertion and hence potential trauma to the wall of the duct of the cochlea receiving the member. The use of a stiffening element or stylet also provides the surgeon with a means to at least partially control the rate of curvature formation in a cochlear electrode assembly during implantation of the elongate carrier members of aspects three to five into the cochlea. Such increased control is envisaged to reduce the potential for trauma to the cochlea caused by electrode assembly insertion.

In a sixth aspect, the present invention is a method of implanting the implantable tissue stimulating device of the first aspect, said method including the steps of:

(i) accessing the implantation site; and

(ii) advancing the elongate carrier member into the cochlea whilst using the one or more optic fibres to illuminate and/or visualise a region of the interior cochlea.

In a seventh aspect, the present invention comprises a method of implanting the cochlear electrode assembly devices of aspects two or three or the cochlear electrode array of aspect four in a body of an implantee, said method including the steps of:

(i) accessing the implantation site; and

(ii) advancing the elongate carrier member into the cochlea.

While advancing the elongate carrier member, the surgeon is able to use the optic fibre stiffening member to illuminate and visualise the region of the cochlea adjacent the distal end of the elongate carrier member. Prior to insertion, the elongate carrier member is preferably substantially straight or straight. Following insertion, the elongate carrier member can adopt a second, preferably spirally curved, configuration.

Once implanted, the electrodes of the elongate carrier member can receive stimulation signals from a stimulator means. The stimulator means is preferably electrically connected to the elongate carrier member by way of an electrical lead. The lead can include the one or more wires extending from each electrode mounted on the elongate carrier member.

In one embodiment, the lead can extend from the elongate carrier member to the stimulator means or at least the housing thereof. In one embodiment, the lead is continuous with no electrical connectors, at least external the housing of the stimulator means, required to connect the wires extending from the electrodes to the stimulator means. One advantage of this arrangement is that there is no requirement for the surgeon implanting the device to make the necessary electrical connection between the wires extending from the electrodes and the stimulator means.

The stimulator means is preferably positioned within a housing that is implantable within the implantee. The housing for the stimulator means is preferably implantable within the bony well in the bone behind the ear posterior to the mastoid.

When implantable, the housing preferably contains, in addition to the stimulator means, a receiver means. The receiver means is preferably adapted to receive signals from a controller means. The controller means is, in use, preferably mounted external to the body of the implantee such that the signals are transmitted transcutaneously through the implantee.

Signals can preferably travel from the controller means to the receiver means and vice versa. The receiver means can include a receiver coil adapted to receive radio frequency (RF) signals from a corresponding transmitter coil worn externally of the body. The radio frequency signals can comprise frequency modulated (FM) signals. While described as a receiver coil, the receiver coil can preferably transmit signals to the transmitter coil which receives the signals.

The transmitter coil is preferably held in position adjacent the implanted location of the receiver coil by way of respective attractive magnets mounted centrally in, or at some other position relative to, the coils.

The external controller can comprise a speech processor adapted to receive signals output by a microphone. During use, the microphone is preferably worn on the pinna of the implantee, however, other suitable locations can be envisaged, such as a lapel of the implantee's clothing. The speech processor encodes the sound detected by the microphone into a sequence of electrical stimuli following given algorithms, such as algorithms already developed for cochlear implant systems. The encoded sequence is transferred to the implanted stimulator/receiver means using the transmitter and receiver coils. The implanted stimulator/receiver means demodulates the FM signals and allocates the electrical pulses to the appropriate attached electrode by an algorithm which is consistent with the chosen speech coding strategy.

The external controller further comprises a power supply. The power supply can comprise one or more rechargeable batteries. The transmitter and receiver coils are used to provide power via transcutaneous induction to the implanted stimulator/receiver means and the electrode array.

While the implant system can rely on external componentry, in another embodiment, the controller means, including the microphone, speech processor and power supply can also be implantable. In this embodiment, the controller means can be contained within a hermetically sealed housing or the housing used for the stimulator means.

According to an eighth aspect, the present invention is a probe for use in the internal visual inspection of a cochlea, the probe comprising an elongate carrier member adapted to be at least partially inserted into one of the ducts of the cochlea and having a proximal end, a distal end, and a lumen formed therein, said lumen extending from a location that is at or adjacent the proximal end at least towards the distal end of the elongate carrier member, the probe further including one or more optic fibres removably positioned within at least a portion of the lumen of the elongate carrier member.

In one embodiment of the eighth aspect, the lumen can extend from the proximal end of the elongate carrier member to a location that is at or adjacent the distal end of the elongate carrier member. In this and other embodiments, the lumen preferably has a first end at or adjacent the proximal end of the elongate carrier member and a second end that is at or adjacent the distal end of the elongate carrier member.

The elongate carrier member may also include a tip member said tip member extending forwardly from the distal end of the elongate carrier member.

In one embodiment of the eighth aspect, the second end of the lumen can be open. In another embodiment, the second end of the lumen can be partially or wholly closed. Where it is closed, the second end of the lumen can be closed by a light permeable member. Further, the light permeable member can comprise one or more lenses that allow visualisation of a region at least adjacent the distal end of the elongate carrier member. The lens can also act as the tip member for the elongate carrier.

In one embodiment, the tip member can be hemispherical in form. The diameter of the tip member preferably gradually decreases to form a rounded end. In one embodiment, the tip member acts as a planar convex lens, however, other lens types can be formed, such as lenses adapted to provide a wide angle or fish eye view of the interior of the cochlea.

In one embodiment, the tip member is formed from a transparent silicone compound. The tip member can further be formed of a material that is substantially the same or is the same stiffness as the body of the elongate carrier member. In another embodiment, the tip member can be formed of a material that is relatively less stiff than at least a portion of the elongate carrier member. In a further embodiment, the tip member can be formed of a material that undergoes a change in stiffness, preferably a decrease in stiffness, on exposure to bodily fluids.

In a further embodiment, the stiffness of the tip member can vary along at least a portion of its length from a first end adjacent the distal end of the elongate carrier member to a second end. In one embodiment, the stiffness of the tip member can vary over the entire length of the tip member or only a portion thereof. The stiffness can increase from the second end to the first end. In one embodiment, the stiffness of the tip member over said portion or its entire length can increase gradually from its second end towards to the first end. The increase in stiffness can be substantially smooth.

In a further embodiment of the eighth aspect, the tip member can be formed of the same material as the body of the elongate carrier member. In another embodiment, the tip member can be formed of a different material to that of the elongate carrier member.

The tip member can be formed separately to the elongate carrier member and mounted thereto. For example, the tip member can be adhered to the distal end of the elongate carrier member with a clear adhesive. In another embodiment, the tip member can be integrally formed with the body of the elongate carrier member.

In one embodiment, the elongate carrier member can be substantially straight. In another embodiment, the elongate carrier member can have a degree of curvature.

In a still further embodiment of the eighth aspect, the elongate carrier member can have a first configuration selected to allow said elongate carrier member to be inserted into an implantee's cochlea and a second configuration wherein the elongate carrier member is curved to at least partially match the curvature of a surface of the cochlea. In this embodiment, the elongate carrier member can be preformed from a plastics material with memory and is preformed to the second configuration. In a preferred embodiment, the first configuration is preferably substantially straight. More preferably, the first configuration is straight. In this and other embodiments, the elongate carrier member can be adapted to adopt a spiral configuration when in the second configuration.

In one embodiment, the elongate carrier member can have a constant diameter along at least a portion or all of its length. The diameter can be between about 0.5 mm and about 0.8 mm. In another embodiment, the elongate carrier member can taper in diameter over some or all of its length towards the distal end. In this regard, the diameter might decrease from about 0.8 mm to about 0.5 mm over the length of the taper. In a further embodiment, the elongate carrier member can be comprised of at least two portions, with one being of constant diameter and the other tapering in diameter over its length.

The cross-sectional shape of the elongate carrier member can be the same along its length or it can vary. The cross-sectional shape may be circular or non-circular, such as square, oval, hexagonal, or octagonal.

The length of the portion of the elongate carrier member that is adapted to be inserted into the cochlea is preferably between about 10 mm and about 30 mm. An elongate carrier member having a length of about 10 mm is preferably adapted to be only inserted such that its tip is at or near the back of the basal turn of the cochlea. Elongate carrier members of longer length can preferably be inserted more deeply into the cochlea and extend around the first turn of the cochlea and so allow visualisation of a greater portion of the interior of the cochlea.

In one embodiment, the elongate carrier member can be formed of a relatively rigid material. In this embodiment, the elongate carrier member is preferably only adapted to be inserted into the cochlea for a length of between about 8.5 and about 10 mm, and so not impact on the lateral wall of the cochlea.

In another embodiment, the elongate carrier member can be formed at least in part or entirely from a relatively non-rigid material. In one embodiment, the elongate carrier member can be formed at least in part or entirely from a resiliently flexible material. Such a material may be particularly suited for elongate carrier members that are adapted to be inserted more deeply than about 10 mm into the cochlea and so must curve around the spiral shape of the cochlea and into the first turn thereof without causing any or at least only relatively minimal trauma to the relatively delicate structures of the wall of the cochlea.

In a preferred embodiment, the elongate carrier member is formed from a suitable biocompatible material. In one embodiment, the material can be a silicone, such as a flexible silicone elastomer Silastic. Silastic MDX 4-4210 is an example of one suitable silicone for use in the formation of the carrier member. In another embodiment, the carrier member can be formed from a polyurethane or other similar materials.

It will be appreciated that the elongate carrier member could be formed from a combination of relatively rigid and non-rigid materials or segments. In this embodiment, the relatively rigid portions may be used for those portions of the member that need to be straight when the elongate carrier member is in the cochlea and the relatively non-rigid portions may be used for those portions of the elongate carrier member that are required to extend around turns in the cochlea.

In a preferred embodiment, the surface of at least the intracochlear section of the elongate carrier member is preferably relatively smooth so minimising trauma to the cochlea.

For the relatively non-rigid portions of the elongate carrier member, silicone rubber or other similar plastic or elastomeric materials may be used. For example, polyurethane or polyurethane copolymers could be utilised. In a further embodiment, the relatively non-rigid portion may be formed from a combination of materials, such as a polypropylene spine with a silicone outer layer.

For the relatively rigid portions of the elongate carrier member, relatively rigid polymeric and/or metallic materials could be utilised.

In a further embodiment, one or more depth markers can be formed in or mounted to the exterior surface of the elongate carrier member. The markers are preferably positioned at pre-defined depths from the distal end of the elongate carrier member and so provide the surgeon with an indication of the depth of the elongate carrier member within the cochlea. In one embodiment, a marker can be positioned at or about 8.5 mm from the distal end of the elongate carrier member. By inserting the member to this depth, the distal end will be positioned just short from the back of the basal turn of the cochlea, so ensuring that the surgeon does not cause the distal end to touch the wall of the cochlea.

Instead, or in addition thereto, a marker can be positioned at or about 10 mm from the distal end of the elongate carrier member. By inserting the elongate carrier member to this depth, the distal end will be positioned at about the position of the lateral wall at the back of the basal turn of the cochlea.

Instead, or in addition thereto, a marker can be positioned at or about 15 mm from the distal end of the elongate carrier member. If the surgeon is able to insert the elongate carrier member to this depth, this will indicate that a subsequently inserted intracochlear electrode assembly could be successfully inserted the cochlea of the patient.

In one embodiment, the one or more markers can be moulded into the elongate carrier member and comprise a protuberance or depression in the member. In another embodiment, a coloured silicone may be used. In this case, different colours can be indicative of different depths. In another embodiment, different marker types can be used for markers at different positions, eg. 1 dot or ring for 8.5 mm, two dots or rings for 10 mm and so on. Materials different from that used to make the elongate carrier member, such as platinum or other biocompatible materials, may be embedded in the elongate carrier member to act as the markers.

In one embodiment, the lumen can be moulded in the elongate carrier member during the manufacture of the member. In one alternative process, a pre-moulded tube can be formed and then over-moulded to form the elongate carrier member, with the lumen of the tube becoming the lumen of the elongate carrier member.

In one embodiment, the one or more optic fibres can be inserted into the lumen of the elongate carrier member following manufacture of the member.

As indicated, the second end of the lumen can be open. In this embodiment, the one or more optic fibres are preferably positionable in the lumen such that the leading end(s) of the fibre(s) are aligned with or positioned back from the opening defining the second end of the lumen. This is desirable as extension of the optic fibres out of the second end of the lumen has the potential to damage the delicate tissues of the cochlea as the elongate carrier member is inserted therein.

In one embodiment, the one or more optic fibres may be provided with a protuberance or collar positioned at a predetermined distance from the leading end thereof to ensure that the optic fibres can only be inserted into the lumen for that predetermined length. The position of the collar or protuberance is preferably such that the leading end of the optic fibre is aligned with or recessed back from the second end of the lumen.

In a further embodiment, a marker may be positioned on the optic fibre that provides a visual cue to the desirable maximum depth of insertion of the optic fibre into the lumen of the elongate carrier member. Once inserted to this or another depth, the optic fibre can be held in this position by a fixation device. Examples of suitable fixation devices include a band (such as rubber band) or crimp that can be clamped around the elongate carrier member at or near the proximal end thereof and so compressing the elongate carrier member and its lumen into frictional engagement with the optic fibre in the lumen.

In one embodiment, a single optic fibre can be used to deliver illumination and be used as the device for allowing visualisation of the interior of the cochlea. In another embodiment, a first optic fibre can be used to deliver illumination and a second optic fibre can be used for visualisation of the interior of the cochlea. In yet another embodiment, a plurality of optic fibres can be used to deliver illumination. In a still further embodiment, a plurality of optic fibres can be used for visualisation of the interior of the cochlea.

In one embodiment, the optic fibre can have a diameter of about 0.2 mm, however, other suitable diameters can be envisaged. The diameter of the lumen in the elongate carrier member will be dependent on factors such as the diameter of the optic fibre and the dimensions of the elongate carrier member. In one embodiment, an elongate carrier member may have a diameter of between about 0.5 and 0.8 mm, with the lumen therein having a diameter of about 0.2 mm.

In one embodiment, the optic fibre can be relatively insertable and/or removable from the lumen of the elongate carrier member. In another embodiment, the optic fibre can be non-removably insertable in the lumen. In yet another embodiment, the optic fibre can be mounted or moulded into the elongate carrier member during manufacture of the elongate carrier member and be adapted to normally remain therein.

In a still further embodiment, a length of one or more optic fibres can be mounted or moulded into the elongate carrier member, with the optic fibre extending from its leading end back through the elongate carrier member for a distance.

In one embodiment, the leading end can be positioned at or adjacent the second end of the lumen and extend for a portion of the length of the elongate carrier member back towards the proximal end of the elongate carrier member to an end that is positioned within the lumen of the elongate carrier member. In this embodiment, a further one or more optic fibres can be insertable, when necessary, into the first end of the lumen to form a butt join with the optic fibre within the elongate carrier member.

In a further embodiment, the leading end of the optic fibre can be positioned at or adjacent the second end of the lumen and extend for a portion of the length of the elongate carrier member back towards the proximal end of the elongate carrier member to an end that is positioned at or external the first end of the lumen of the elongate carrier member. In this embodiment, an end of the optic fibre can be connected to an optical fibre termination device. The termination device preferably receives a proximal end of the optic fibre and can have a light source, eyepiece, and/or a camera lens mounted thereto. The termination device preferably receives light output by the light source and directs this light through the one or more optic fibres to their leading ends. This light is then preferably able to pass through the open end of the lumen or the light permeable tip member of the carrier and so illuminate at least the region of the cochlea adjacent the distal end of the elongate carrier member.

The eyepiece and/or the camera lens preferably receive light reflected through the one or more optic fibres from an object illuminated by the light emitting from the distal end of the optic fibres. A magnifying device and/or focusing device can be incorporated, if necessary, into the termination means.

The camera lens is preferably part of a video camera that allows recordal of the image detected by the camera lens. The video footage is preferably used in real time during insertion of the probe into a recipient's cochlea but can also be used as a means of reviewing the surgical procedure following completion of the surgery.

In one embodiment, the elongate carrier member can be designed for single use. In another embodiment, the elongate carrier member can be designed to be sterilisable after use and so be reusable.

The lumen of the elongate carrier member can be adapted to receive a stiffening element removably positionable within said elongate carrier member said stiffening member biasing said elongate carrier member into its first configuration. In this aspect, the elongate carrier member is preferably pre-packaged with a stiffening element positioned within the lumen of the elongate carrier member. The stiffening element is preferably a stylet, such as a platinum stylet or a suitable relatively stiff polymeric material stylet. In another embodiment, the stiffening element can comprise a monolithic optic fibre stylet. In another embodiment, the stiffening element can comprise a stylet formed of a plurality of optic fibres.

In a further embodiment, the elongate carrier member can have a second lumen that is adapted to receive a stiffening element that is removably positionable therein. In yet a further embodiment, the optic fibre can be non-removably mounted or moulded in the elongate carrier member with the stylet removably positioned within the lumen thereof.

During insertion of the elongate carrier member into a cochlea, it is preferred that the elongate carrier member follow a trajectory that is as close as possible to the middle of the lumen of the scala tympani of the cochlea as is possible thereby minimising the potential for trauma. This technique also preferably avoids pressure and potential trauma to the modiolar wall of the cochlea.

In one embodiment, the elongate carrier member is preferably inserted into the cochlea by being advanced off a stylet using a three phase technique. This technique firstly preferably involves introducing the distal end of the elongate carrier member into the cochlea and advancing the elongate carrier member into the cochlea for a distance of approximately 8.5 mm. When at this location, the distal end of the elongate carrier member is preferably near the back of the basal turn of the cochlea. The elongate carrier member is then preferably relatively advanced off the stylet and inserted more deeply into the scala tympani. Once free of the elongate carrier member, the stylet can be withdrawn from the cochlea.

The three phase technique of inserting the elongate carrier member can be performed manually by the physician or surgeon positioning the elongate carrier member in the cochlea. In another embodiment, a tool can be used to appropriately position and then advance the elongate carrier member off the stylet and into the desired location in the cochlea.

In one embodiment of the eighth aspect, the elongate carrier member is preferably adapted to allow visualisation of the interior of the cochlea and then be withdrawn from the cochlea when it is no longer required. The elongate carrier member may, however, also act as a device for the delivery of electrical or other stimulation to the interior of the cochlea. In this latter embodiment, the carrier member may have one or a plurality of electrodes mounted thereon.

In a ninth aspect, the present invention comprises a method of inserting the probe of the eighth aspect in a body of an implantee, comprising the steps of:

(i) accessing the insertion site;

(ii) introducing the distal end of the elongate carrier member into the cochlea and advancing a substantial length of the elongate carrier member into the cochlea.

During the insertion, the surgeon is able to use the optic fibre to illuminate and visualise the region of the cochlea adjacent the tip of the elongate carrier member of the probe. Prior to insertion, the elongate carrier member is preferably substantially straight or straight. Following insertion, the elongate carrier member can, as mentioned, adopt a second, preferably spirally curved, configuration.

In a tenth aspect, the present invention is a probe for use in the internal visual inspection of a cochlea, the probe comprising an elongate carrier member adapted to be at least partially inserted into one of the ducts of the cochlea and having a proximal end, a distal end, and a lumen formed therein extending from a location that is at or adjacent the proximal end at least towards the distal end of the elongate carrier member, the lumen being adapted to receive one or more optic fibres.

### BRIEF DESCRIPTION OF THE DRAWINGS

By way of example only, preferred embodiments of the invention are now described with reference to the accompanying drawings, in which:

FIG. 1 is a simplified cross-sectional view of an embodiment of an electrode assembly according to the present invention depicted in its first configuration that can also be used to visualise the interior of a cochlea;

FIG. 2 is a simplified side elevational view of the electrode assembly of FIG. 1 depicted with the stylet partially withdrawn;

FIG. 3 is a simplified side elevational view of the electrode assembly depicted in FIG. 1 implanted in the cochlea;

FIG. 4 is a simplified schematic view of a light source, eyepiece, and camera for use with the optic fibre of the present invention.

FIG. 5 is a schematic view of one embodiment of a probe for visualising the interior of a cochlea according to the present invention;

FIG. 6 is a schematic view of another embodiment of a probe for visualising the interior of a cochlea according to the present invention; and

FIG. 7 is a schematic view of a still further embodiment of a probe for visualising the interior of a cochlea according to the present invention;

### PREFERRED MODE OF CARRYING OUT THE INVENTION

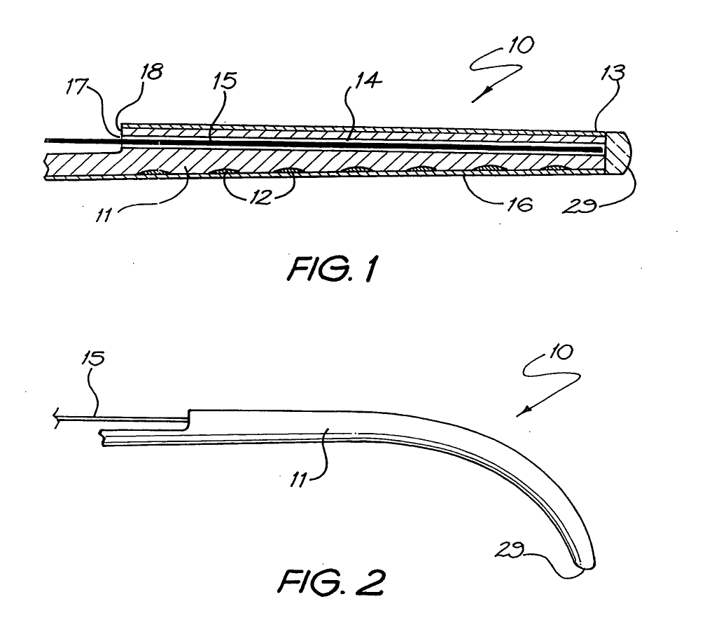

One embodiment of a cochlear implant electrode assembly according to the present invention is depicted generally as 10 in FIGS. 1 to 3.

The depicted electrode assembly 10 has an electrical lead extending back to a receiver/stimulator housing. In considering this invention, it is to be understood that each electrode may have one or more wires (not depicted) electrically connected thereto and extending from each respective electrode back through the lead to the receiver/stimulator.

The assembly 10 comprises an elongate electrode carrier member 11 having a plurality of electrodes 12 mounted thereon. For the purposes of clarity, the electrodes 12 depicted in FIG. 1 are not necessarily shown to scale.

The depicted elongate member 11 is made from a resiliently flexible silicone suitable for insertion in the scala tympani of the cochlea. The elongate member 11 has a first end 13 that is firstly inserted into the implantee on insertion of the assembly 10.

The elongate member 11 has a partly hemispherical tip member 29 integrally formed with its first end 13. The depicted tip 29 is formed from a transparent silicone and has a resilient flexibility substantially equal to that of the material used for the carrier member 11. The depicted tip member is transparent and acts as a lens so that light delivered to the first end 13 of the carrier member can illuminate at least the region of the duct of the cochlea adjacent the first end 13 when the member 11 is in the duct.

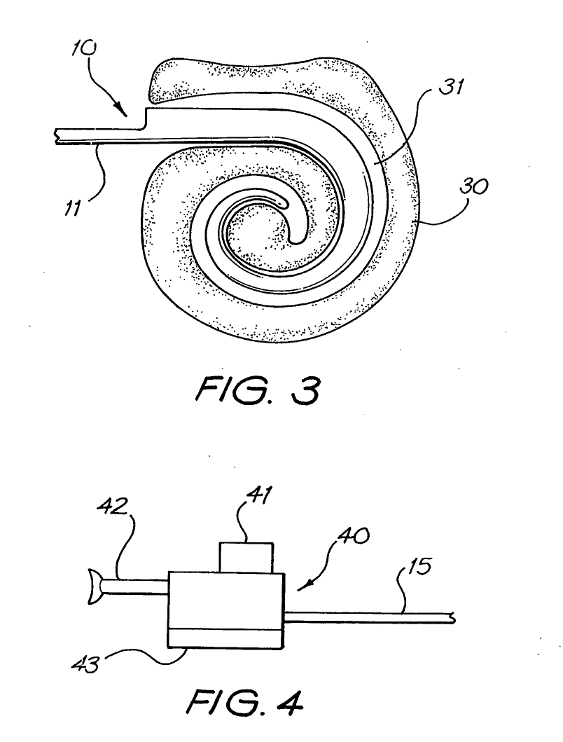

Disposed within a substantially cylindrical lumen 14 extending the length of carrier member 11 is an optic fibre element 15. The optic fibre element 15 extends through opening 17 in lumen 14 to a termination apparatus 40 depicted schematically in FIG. 4. The apparatus 40 receives the proximal end of the optic fibre element 15. It comprises a light source 41, eyepiece 42, and a video camera 43. Light from the light source 41 is reflected within the apparatus 40 and transmitted through the optic fibre element 15 to its distal end where the light can then exit the elongate member 11 through transparent tip 29 and illuminate the region of the cochlea adjacent the tip 29.

The eyepiece 42 and video camera 43 receive light reflected from objects illuminated by the tip of the distal end of the optic fibre element 15 and allow examination thereof. While the termination apparatus 40 is depicted as having both an eyepiece 42 and camera 43, one of these devices could be omitted if desired.

On insertion of the device 10 into the scala tympani of the cochlea 30, the surgeon can visualise the duct of the cochlea through the eyepiece 42 or output of the camera 43, with the duct being illuminated by light transmitted into the cochlea via the optic fibre element 15 and transparent tip member 29. This illumination and visualisation allows the surgeon to note and, if possible, avoid obstructions in the duct during the insertion process. It also provides the surgeon with an ability to determine if the insertion technique has caused any damage to the sensitive structures of the cochlea, and to avoid the likelihood of such damage occurring.

The optical fibre element 15 provides the surgeon with greater control of the implantation procedure for the cochlear implant electrode assembly 10. The provision of greater control minimises the potential for trauma to the sensitive tissues inside the cochlea and also enhances the likelihood of successful placement of the assembly 10 at the first attempt.

The optical fibre element 15 can consist of a plurality of optical fibre elements whereby some of the fibre elements are used to carry light along the length of the electrode assembly 10 and other optic fibre elements are used to capture the optic signal and relay this back to the termination apparatus 40.

The lumen 14 and optic fibre element 15 can be provided in a wall of the electrode assembly separate to an additional lumen or stiffening stylet that could be employed in a conventional manner to straighten a pre-curved electrode assembly for insertion.

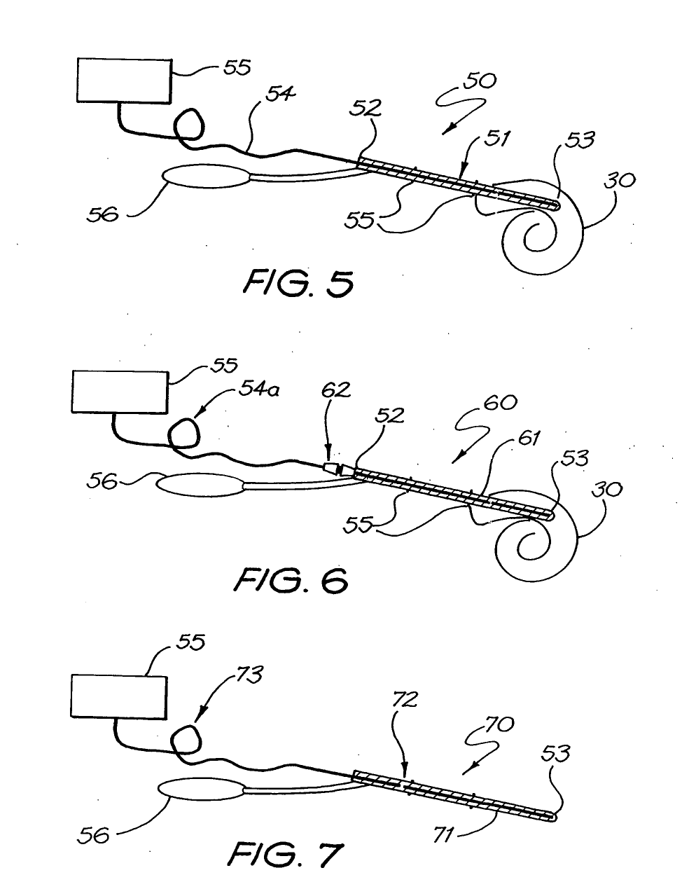

However, as is shown in FIGS. 5 and 6, the lumen 14 and optic fibre element could act as a conventional stiffening stylet as will be described below.

In this embodiment, the optic fibre element 15 disposed within lumen 14 acts as a stylet and is relatively stiffer than the elongate carrier 11 and has a stiffness that is sufficient to retain the silicone elongate member 11 in the straight configuration depicted in FIG. 1 during the insertion procedure of the member 11 in the cochlea of the recipient.

The optic fibre element stylet stylet 15 extends through opening 17 in lumen 14 to a termination apparatus 40 depicted schematically in FIG. 4 in the same manner as described above.

While the elongate member 11 is manufactured with a preformed curved configuration, the assembly 10 is typically delivered to a surgeon with the optic fibre element stylet 15 in place. The placement of the optic fibre element stylet 15 in the lumen 14 is sufficient to hold the elongate member 11 in the straight configuration depicted in FIG. 1.

On insertion of the device 10 into the scala tympani of the cochlea 30, the surgeon can visualise the duct of the cochlea through the eyepiece 42 or output of the camera 43, with the duct being illuminated by light transmitted into the cochlea via the optic fibre element stylet 15 and transparent tip member 29. This illumination and visualisation allows the surgeon to note and, if possible, avoid obstructions in the duct during the insertion process. When the first end 13 reaches the back of the basal turn, the surgeon can commence withdrawal of the optic fibre element stylet 15 from the lumen 14. This can be achieved by gripping and withdrawing the optic fibre element stylet 15 or by moving the termination apparatus 40, with the optic fibre element stylet 15 connected thereto, relatively away from the recipient's cochlea. As the optic fibre element stylet 15 is withdrawn, the elongate member 11 commences to re-curl (see FIG. 2).

As the elongate member 11 curls, the surgeon can continue to further insert the curled assembly 10 into the scala tympani duct until the desired insertion is attained. Upon desired insertion, the optic fibre element stylet 15 can be fully withdrawn through the opening 17 of the lumen 14. On full withdrawal of the optic fibre element stylet 15, the elongate member 11 is free to adopt the spiral configuration depicted in FIG. 6 with the electrodes 12 facing the modiolus within the cochlea 30 so that they are positioned as close as possible to the spiral ganglia thereof.

The optic fibre element stylet 15 provides the surgeon with greater control of the implantation procedure for the cochlear implant electrode assembly 10. The provision of greater control minimises the potential for trauma to the sensitive tissues inside the cochlea and also enhances the likelihood of successful placement of the assembly 10 at the first attempt.

In this regard it is envisaged that the optic fibre element stylet 15 can consist of more than one element having potentially two functions, or redundant functions. For example, in one embodiment the optic fibre element stylet 15 can consist of two elements, one element to perform the stiffening function, and one optic fibre element to perform the optical function and supplement the stiffening of the electrode array. In another embodiment, two optic fibre elements may be provides where one element acts as a light source transporting light to the end of the electrode array, and the other captures the optic signal, with both elements together acting to perform the stiffening function. It is also envisaged that two identical optic fibre elements can be provided, with both element functioning to perform the optics and some stiffening. In this embodiment, if there are two such optic fibre elements, then an electrode array can be provided having 3 different configurations (straight, partly curved and fully curved), by removing one of the elements at different points in the insertion procedure. Such an embodiment may be useful for providing an atraumatic insertion procedure.

While the preferred embodiment of the invention has been described in conjunction with a cochlear implant, it is to be understood that the present invention has wider application to other implantable electrodes, such as electrodes used with pacemakers.

In another embodiment of the present invention, rather than incorporating the optic fibre feature into an intracochlear electrode array, a probe is provided for use in the visualisation of the interior of the cochlea and which is then withdrawn from the cochlea when it is no longer required is depicted generally as 50 in FIG. 5.

The probe 50 comprises a straight elongate carrier member 51 that is adapted to be at least partially inserted into one of the ducts (eg. the scala tympani) of the cochlea 30. The member 51 has a proximal end 52, a distal end 53, and a lumen formed therein that extends from the proximal end 51 of the member to a location that is at or adjacent the distal end 53 of the carrier member 51. As depicted in FIG. 5, the lumen is adapted to receive one or more optic fibres 54 that extend from an endoscopic system 55 and into the lumen.

In the embodiment depicted in FIG. 5, the second end of the lumen at the distal end 53 of the member 51 is open. In another embodiment, the second end of the lumen can be partially or wholly closed by a tip. The tip can be a light permeable member and comprise one or more lenses that allow visualisation of a region at least adjacent the tip of the carrier member 51. In one embodiment, the tip member acts as a planar convex lens, however, other lens types can be formed, such as lenses adapted to provide a wide angle or fish eye view of the interior of the cochlea.

While member 51 is depicted as being straight, it will be appreciated that the carrier member can be fabricated to have a degree of curvature. As is described above in relation to the embodiment depicted in FIG. 1, the carrier member 51 can also have a first straight configuration selected to allow the member to be inserted into an implantee's cochlea and a second configuration wherein the member is spirally curved to at least partially match the curvature of a surface of the cochlea.

The carrier member 51 has a constant diameter along at least a portion of its length. The diameter can vary but can be between about 0.5 mm and about 0.8 mm.

While not depicted, the carrier member 51 could be manufactured so as to taper in diameter over some or all of its length towards the distal end 53. In this regard, the diameter might decrease from about 0.8 mm to about 0.5 mm over the length of the taper. In a further embodiment, the carrier member can be comprised of at least two portions, with one being of constant diameter and the other tapering in diameter over its length.

The cross-sectional shape of the carrier member 51 is circular and constant along its length. It will be appreciated that the cross-sectional shape could vary along its length and be non-circular, such as square, oval, hexagonal, or octagonal.

The portion of the carrier member 51 that is adapted to be inserted into the cochlea is preferably between about 10 mm and about 30 mm in length. A member having a length of about 10 mm is preferably adapted to be only inserted such that its tip is at or near the back of the basal turn of the cochlea 30. Carrier members of longer length can preferably be inserted more deeply into the cochlea and extend around the first turn of the cochlea 30 and so allow visualisation of a greater portion of the interior of the cochlea 30.

The depicted carrier member 51 is formed from a suitable biocompatible material, such as a flexible silicone elastomer Silastic. Silastic MDX 4-4210 is an example of one suitable silicone for use in the formation of the carrier member. In another embodiment, the carrier member can be formed from a polyurethane or other similar materials.

It will be appreciated that the carrier member 51 could be formed from a combination of relatively rigid and non-rigid materials or segments. The surface of at least the intracochlear section of the carrier member 51 is also preferably relatively smooth so minimising trauma to the cochlea 30.

As depicted in FIG. 5, the carrier member is provided with a set of depth markers 55 that are formed in or mounted to the exterior surface of the carrier member 51. The markers 55 are positioned at pre-defined depths from the distal end 53 of the member 51 and so provide the surgeon with an indication of the depth of the member 51 within the cochlea 30. In the depicted embodiment, a marker is positioned at or about 8.5 mm from the distal end 53 of the carrier member 51. By inserting the member to this depth, the distal end will be positioned just short from the back of the basal turn of the cochlea, so ensuring that the surgeon does not cause the distal end to touch the wall of the cochlea 30, as is depicted in FIG. 5.

In addition thereto, a marker is positioned at or about 15 mm from the distal end 53 of the member 51. If the surgeon was able to insert the member 51 to this depth, this would indicate that a subsequently inserted intracochlear electrode assembly could be successfully inserted the cochlea 30 of the patient.

In the depicted embodiment, the markers 55 comprise a moulded protuberance in the member 51. In another embodiment, not depicted, a coloured silicone could be used as the marker. In this case, different colours can be indicative of different depths. In another embodiment, different marker types can be used for markers at different positions, eg. 1 dot or ring for the first marker two dots or rings for the second marker and so on. Materials different from that used to make the carrier member, such as platinum or other biocompatible materials, may also be embedded in the member to act as the markers.

As depicted, the member 51 can be provided with a handle 56 that allows the surgeon to more readily manipulate the member 51 during implantation in the cochlea 30 of the patient.

A number of techniques can be used to form the lumen in the member 51. In one method, the lumen can be moulded in the carrier member 51 during the manufacture of the member 51. In one alternative process, a pre-moulded tube can be formed and then over-moulded to form the member 51, with the lumen of the tube becoming the lumen of the member 51.

FIG. 5 depicts an arrangement in which the optic fibre or fibres 54 are inserted into the lumen of the member following manufacture of the member 51. For example, the fibres 54 may be inserted by the surgeon just prior to or during insertion of the member 51 into the cochlea 30.

As indicated, the second end of the lumen of member 51 is open. In this embodiment, the one or more optic fibres 54 are preferably positionable in the lumen such that the leading end(s) of the fibre(s) are aligned with or positioned back from the opening defining the second end of the lumen. This is desirable as extension of the optic fibres out of the second end of the lumen has the potential to damage the delicate tissues of the cochlea 30 as the member 51 is inserted therein.

In one embodiment, the one or more optic fibres 54 may be provided with a protuberance or collar positioned at a predetermined distance from the leading end thereof to ensure that the optic fibres 54 can only be inserted into the lumen for that predetermined length. The position of the collar or protuberance is preferably such that the leading end of the optic fibre 54 is aligned with or recessed back from the second end of the lumen. Other pre-defined desired lengths of insertion of the optic fibres into the carrier member 51 can be envisaged.

In a further embodiment, a marker may be positioned on the optic fibre 54 that provides a visual cue to the desirable maximum depth of insertion of the optic fibre into the lumen of the carrier member 51. Once inserted to this or another depth, the optic fibre 54 can be held in this position by a fixation device. Examples of suitable fixation devices include a band (such as rubber band) or crimp that can be clamped around the carrier member 51 at or near the proximal end 52 thereof and so compressing the carrier member 51 and its lumen into frictional engagement with the optic fibre 54 in the lumen.

In one embodiment, a single optic fibre can be used to deliver illumination and be used as the device for allowing visualisation of the interior of the cochlea. In another embodiment, a first optic fibre can be used to deliver illumination and a second optic fibre can be used for visualisation of the interior of the cochlea. In yet another embodiment, a plurality of optic fibres can be used to deliver illumination. In a still further embodiment, a plurality of optic fibres can be used for visualisation of the interior of the cochlea.

In one embodiment, the optic fibre can have a diameter of about 0.2 mm, however, other suitable diameters can be envisaged. The diameter of the lumen in the carrier member will be dependent on factors such as the diameter of the optic fibre and the dimensions of the carrier member. In one embodiment, a carrier member may have a diameter of between about 0.5 and 0.8 mm, with the lumen therein having a diameter of about 0.2 mm.

In FIG. 5, the optic fibre 54 is relatively insertable and/or removable from the lumen of the carrier member 51. In another embodiment, the optic fibre can be non-removably insertable in the lumen. In yet another embodiment, the optic fibre can be mounted or moulded into the carrier member during manufacture of the carrier member and be adapted to normally remain therein, as is depicted in FIGS. 6 and 7. In these figures, like features of the probe of FIG. 5 are identified using the same reference numerals as that used for FIG. 5.

In FIG. 6, a length of one or more optic fibres 61 is mounted or moulded into the carrier member 60, with the optic fibre 61 extending from its leading end located at the distal end 53 back through the carrier member 60 for a distance. In this embodiment, the leading end of the optic fibre 61 is positioned at or adjacent the second end of the lumen and extends for a portion of the length of the carrier member 60 back towards the proximal end 52 of the carrier member 60 to an end that is positioned at or external the first end of the lumen of the carrier member 60. In this embodiment, an end of the fibre optic can be connected to an optical fibre connector 62 that in turn is connected to a complementary connector mounted to one or more optic fibres 54a extending from the endoscopic device 55. The connector 62 allows the optic fibres 54a extending from the endoscopic system 55 to be connected to the optic fibre 61 within the carrier member 60 when necessary.

In the embodiment depicted in FIG. 7, the leading end of an optic fibre 71 can be positioned at or adjacent the second end of the lumen and extend for a portion of the length of the carrier member 70 back towards the proximal end 52 of the carrier member 70 to an end that is positioned well within the lumen of the carrier member 70. In this embodiment, a further one or more optic fibres 73 can extend from the endoscopic system 55 and be insertable, when necessary, into the first end of the lumen to form a butt join 72 with the optic fibre 71 within the carrier member 70.

The depicted endoscopic system 55 can have a light source, eyepiece, and/or a camera lens mounted thereto. The light output by the light source is directed through the one or more optic fibres to their distal ends. This light is then preferably able to pass through the open end of the lumen of the carrier and so illuminate at least the region of the cochlea adjacent the distal end of the carrier member being used.

The eyepiece and/or the camera lens preferably receive light reflected through the one or more optic fibres from an object illuminated by the light emitting from the distal end of the optic fibres. A magnifying device and/or focussing device can be incorporated, if necessary, into the endoscopic system 55.

The camera lens is preferably part of a video camera that allows recordal of the image detected by the camera lens. The video footage is preferably used in real time during insertion of the probe into a recipient's cochlea but can also be used as a means of reviewing the surgical procedure following completion of the surgery.

In one embodiment, the carrier member can be designed for single use. In another embodiment, the carrier member can be designed to be sterilisable after use and so be reusable.

The lumens of the respective carrier members can be adapted to receive a stiffening element that is removably positionable within the member and which biases the carrier member into the mentioned straight first configuration. In this aspect, the carrier member device is preferably pre-packaged with a stiffening element positioned within the lumen of the member. The stiffening element is preferably a stylet, such as a platinum stylet or a suitable relatively stiff polymeric material stylet. In another embodiment, the stiffening element can comprise a monolithic optic fibre stylet. In another embodiment, the stiffening element can comprise a stylet formed of a plurality of optic fibres.

In another embodiment, the carrier member can have a second lumen that is adapted to receive a stiffening element that is removably positionable therein. In yet a further embodiment, the optic fibre can be non-removably mounted or moulded in the body of the carrier member with the stylet removably positioned within the lumen thereof.

During implantation of the carrier member, it is preferred that the carrier member follow a trajectory that is as close as possible to the middle of the lumen of the scala tympani of the cochlea as is possible thereby minimising the potential for trauma. This technique also preferably avoids pressure and potential trauma to the modiolar wall of the cochlea.

In one embodiment, the carrier member is preferably inserted into the cochlea by being advanced off a stylet using a three phase technique. This technique firstly preferably involves introducing the distal end of the carrier member into the cochlea and advancing the member into the cochlea for a distance of approximately 8.5 mm. When at this location, the distal end of the carrier member is preferably near the back of the basal turn of the cochlea. The member is then preferably relatively advanced off the stylet and inserted more deeply into the scala tympani. Once free of the member, the stylet can be withdrawn from the cochlea.

The three phase technique of inserting the carrier member can be performed manually by the physician or surgeon positioning the carrier member in the cochlea. In another embodiment, a tool can be used to appropriately position and then advance the carrier member off the stylet and into the desired location in the cochlea.

As mentioned, the probe 50 of FIG. 5 and the alternative embodiments depicted in FIGS. 6 and 7 are adapted to be used prior to the implantation of a cochlear implant electrode assembly.

While the preferred embodiment of the invention has been described in conjunction with a cochlear implant, it is to be understood that the present invention has wider application to other implantable electrodes, such as electrodes used with pacemakers.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An implantable tissue stimulating device comprising an elongate carrier member having a proximal end, a distal end, and a plurality of electrodes mounted thereon between said proximal and distal ends, the elongate carrier member having at least a first lumen extending at least partially therethrough, the elongate carrier member further including one or more optic fibres positioned along a length of said at least a first lumen.

2. The implantable tissue stimulating device of claim 1 comprising a cochlear electrode array.

3. The implantable tissue stimulating device of claim 2 wherein the one or more optic fibres allow a user to illuminate and/or visualise an area of the cochlea during or prior to surgery and wherein further, said one or more optic fibres are removably positioned within said at least a first lumen.

4. The implantable tissue stimulating device of claim 1 wherein the elongate carrier member includes a plurality of optic fibres.

5. The implantable tissue stimulating device of claim 4 wherein at least one of said plurality of optic fibres allows illumination of a surgical site and at least a further optic fibre allows a user to visualise said surgical site.

6. The implantable tissue stimulating device of claim 2 wherein the elongate carrier member has a first configuration selected to allow said elongate carrier member to be inserted into the cochlea, and at least a second configuration wherein said elongate carrier member is adapted to apply a preselected tissue stimulation with the electrodes, said elongate carrier member being made of a resiliently flexible first material.

7. The implantable tissue stimulating device of claim 6 wherein the one or more optic fibres act as a stiffening element that biases said elongate carrier member into said first configuration and wherein removal of the stiffening element causes the elongate carrier member to assume its second configuration.

8. The implantable tissue stimulating device of claim 6 wherein the elongate carrier member includes a second lumen to receive a stiffening element said stiffening element biasing the elongate carrier member into its first configuration.

9. The implantable tissue stimulating device of claim 3 wherein upon removal of the one or more optic fibres, the at least a first lumen acts as a drug delivery channel.

10. The implantable tissue stimulating device of claim 1 wherein the elongate carrier member has a resiliently flexible tip member extending forwardly from the distal end of the elongate carrier member, said tip member being light permeable and hemispherical in form.

11. The implantable tissue stimulating device of claim 10 wherein the tip member acts as a lens and allows illumination and/or visualisation of a region at least adjacent the tip member of the elongate carrier member.

12. The implantable tissue stimulating device wherein the one or more optic fibre is connected to an optical fibre termination means said optical fibre termination means including a light source, eyepiece, and/or a camera lens mounted thereto wherein further, said optical fibre termination means receives light output by the light source and directs this light through the one or more optic fibres.

13. An implantable tissue-stimulating device comprising:
an elongate carrier member having a plurality of electrodes mounted thereon and having a first configuration selected to allow said elongate carrier member to be inserted into an implantee's body, and at least a second configuration wherein said elongate carrier member is adapted to apply a preselected tissue stimulation with the electrodes, said elongate carrier member being made of a resiliently flexible first material; and
a stiffening element removably positionable within said elongate carrier member that biases said elongate carrier member into said first configuration;
wherein said stiffening element comprises one or more optic fibres.

14. The implantable tissue-stimulating device of claim 13 wherein the elongate carrier member extends from a proximal end to a distal end and has a resiliently flexible tip member extending forwardly from the distal end of the elongate carrier member, said tip member being light permeable and hemispherical in form.

15. A cochlear implant electrode assembly device comprising:
an elongate carrier member having a plurality of electrodes mounted thereon and having a first configuration selected to allow said elongate carrier member to be inserted into an implantee's cochlea, and at least a second configuration wherein said elongate carrier member is curved to match a surface of said cochlea, said elongate carrier member being made of a resiliently flexible first material; and
a stiffening element removably positionable within said elongate carrier member that biases said elongate carrier member into said first configuration;
wherein said stiffening element comprises one or more optic fibres.

16. The cochlear implant electrode assembly device of claim 15 wherein the elongate carrier member extends from a proximal end to a distal end and wherein the elongate carrier member has a resiliently flexible tip member extending forwardly from the distal end of the elongate carrier member, said tip member being light permeable and hemispherical in form.

17. A cochlear implant electrode assembly device comprising an elongate carrier member having a proximal end, a distal end, and a plurality of electrodes mounted thereon between said proximal and distal ends, the elongate carrier member having a first configuration selected to allow said member to be inserted into an implantee's cochlea, and at least a second configuration wherein said elongate carrier member is curved to match a surface of said cochlea, said elongate carrier member being made of a resiliently flexible first material and having a lumen formed therein extending from or adjacent the proximal end to or adjacent the distal end and adapted to receive a stiffening element removably positionable within said elongate carrier member that biases said elongate carrier member into said first configuration, wherein said distal end of said elongate carrier member comprises a transparent tip member.

18. The cochlear implant electrode assembly device of claim 17 wherein said assembly device is pre-packaged with the stiffening element positioned within the lumen of the member.

19. A method of implanting the implantable tissue stimulating device of claim 1, said method including the steps of:
(i) accessing the implantation site; and
(ii) advancing the elongate carrier member into the cochlea whilst using the one or more optic fibres to illuminate and/or visualise a region of the interior cochlea.

20. The method of claim 19 wherein a surgeon manipulates the elongate carrier member to avoid trauma to the tissues of the cochlea.

21. The method of claim 19 wherein once implanted, the electrodes of the elongate carrier member receive stimulation signals from a stimulator means said stimulator means electrically connected to the elongate carrier member by way of an electrical lead.

22. The method of claim 21 wherein the stimulator means is positioned within a housing that is implanted within the implantee and wherein the housing contains in addition to the stimulator means, a receiver means to receive signals from a controller means, said controller means mounted external to the body of the implantee such that the signals are transmitted transcutaneously through the implantee.

23. The method of claim 22 wherein the signals travel from the controller means to the receiver means and vice versa.

24. A method of implanting the cochlear electrode assembly device of claim 15, said method including the steps of:
(i) accessing the implantation site; and
(ii) advancing the elongate carrier member into the cochlea.

25. The method of claim 24 wherein as the elongate carrier member is advanced into the cochlea, the surgeon is uses the optic fibre stiffening member to visualise a region of the cochlea.

26. A stiffening element for an implantable tissue-stimulating device characterised in that the stiffening element comprises one or more optic fibres.

27. A probe for use in the internal visual inspection of a cochlea, the probe comprising an elongate carrier member adapted to be at least partially inserted into one of the ducts of the cochlea and having a proximal end, a distal end, and a lumen formed therein, said lumen extending from a location that is at or adjacent the proximal end at least towards the distal end of the elongate carrier member, the probe further including one or more optic fibres removably positioned within at least a portion of the lumen of the elongate carrier member.

28. The probe of claim 27 wherein the lumen extends from a first end to a second end and wherein the first end is at or adjacent the proximal end of the elongate carrier member and the second end is at or adjacent the distal end of the elongate carrier member.

29. The probe of claim 27 wherein the second end of the lumen is open.

30. The probe of claim 27 wherein the second end of the lumen is either partially or wholly closed.

31. The probe of claim 30 wherein the lumen is partially or wholly closed by a light permeable member, said light permeable member comprising one or more lenses that allow visualisation of a region at least adjacent the distal end of the elongate carrier member.

32. The probe of claim 31 wherein the one or more lenses act(s) as a tip member for the elongate carrier member said tip member extending forwardly from the distal end of the elongate carrier member.

33. The probe of claim 27 wherein the elongate carrier member has a first configuration selected to allow said elongate carrier member to be inserted into an implantee's cochlea and a second configuration wherein the elongate carrier member is curved to at least partially match the curvature of a surface of the cochlea.

34. The probe of claim 33 wherein the first configuration is substantially straight and when in the second position, the elongate carrier member adopts a spiral configuration.

35. A method of inserting the probe of claim 27 in a body of an implantee, comprising the steps of:
(i) accessing the insertion site; and
(ii) introducing the distal end of the elongate carrier member into the cochlea and advancing a substantial length of the elongate carrier member into the cochlea.

36. The method of claim 35 wherein during insertion of the elongate carrier member, a surgeon uses the one or more optic fibres to illuminate and visualise the region of the cochlea adjacent the distal end of the elongate carrier member.

37. The method of claim 35 wherein the probe includes a stiffening member comprising a stylet and wherein during step (ii), the distal end of the elongate carrier member is advanced to a position near the back of the basal turn of the cochlea and subsequently advanced off the stylet such that the distal end of the elongate carrier member is inserted relatively deeper into the scala tympani.

38. (canceled)
